SEQUENCE LISTING (4 sequences)

SEQ ID NO: 1 - DNA, 21 bp, Artificial
  Artificial primer sequence
acggccgcgacgacagccaca

SEQ ID NO: 2 - DNA, 21 bp, Artificial
  Artificial primer sequence
gctgcgggtccacatctctgc

SEQ ID NO: 3 - DNA, 21 bp, Artificial
  Artificial primer sequence
aaaccagaggagataaaaatg

SEQ ID NO: 4 - DNA, 21 bp, Artificial
  Artificial primer sequence
gaatgagagtgcttgtgtgag

(19) United States
(12) Patent Application Publication
(10) Pub. No.: US 2008/0318216 A1
(43) Pub. Date: Dec. 25, 2008

(54) Methods For Determining Presence of Cancer by Analyzing the Expression of Cdk9 and/or Cyclin T1 in Lymphoid Tissue

(76) Inventors: Antonio Giordano, Radnor, PA (US); Piero Tosi, Siena (IT)

Correspondence Address: REED SMITH LLP, 2500 ONE LIBERTY PLACE, 1650 MARKET STREET, PHILADELPHIA, PA, 19103, US

(21) Appl. No.: 11/632,244

(22) PCT Filed: Jul. 12, 2005
(86) PCT No.: PCT/US05/24585

§ 371 Date: Oct. 24, 2007

Related U.S. Application Data
(60) Provisional application No. 60/587,213, filed on Jul. 12, 2004.

(51) Int. Cl. C12Q 1/68; G01N 33/00; C12Q 1/00; G01N 33/574

(52) U.S. Cl. 435/6; 436/64; 435/4; 435/7.23

(57) ABSTRACT

A method for determining presence of lymphoma in a patient is disclosed. A sample of bone marrow, thymus, spleen, lymph nodes, lymph and/or lymphocytes taken from the patient t is assayed to determine expression of CDK9 and CYCLIN T1. The presence of CDK9 and/or CYCLIN T1 is indicative of a lymphoma other than a mantle cell lymphoma or marginal zone lymphoma in the patient.

This application claims the benefit of U.S. Provisional Application No. 60/587,213 filed Jul. 12, 2004, the text of which application is hereby incorporated by reference in its entirety.

### FIELD OF THE INVENTION

The present invention relates to expression patterns of CDK9 and CYCLIN T1 in malignant lymphocytes or lymphomas and methods of diagnosis of lymphomas or identification of occult tumour contamination in the autologous bone marrow based on the CDK9 and CYCLIN T1 expression, and treatment of lymphomas.

### BACKGROUND OF THE INVENTION

Lymph system is a network of organs and nodes that interacts with the circulatory system to transport a watery clear fluid called lymph throughout the body. Lymph contains cells called lymphocytes. There are two main types of lymphocytes: B-cells and T-cells. The B-cells originate from stem cells in the bone marrow and complete their structural growth (differentiation) and mature in the bone marrow. The T-cells also start out in the bone marrow, but they differentiate and mature in the thymus gland. The B-cell and T-cell lymphocytes leave these organs through the blood stream. They then migrate to different parts of the body and perform unique functions at each stage.

Lymphomas are a group of related cancers that arise when lymphocytes become malignant. When a cell becomes malignant its maturation stage is arrested and the developmental stage of a lymphocyte when it becomes malignant determines the specific kind of lymphoma. There are different subtypes and maturation stages of lymphocytes and, therefore, there are different kinds of lymphomas. Lymphomas are generally subdivided into two groups; classical Hodgkin's lymphoma (Hodgkin's disease) and non-Hodgkin's lymphomas. Like normal cells, malignant lymphocytes can move to many parts of the body.

Lymphomas are difficult to diagnose and no single test is currently sufficient to establish the diagnosis of lymphomas. Rather, current clinical practice involves a pathologist looking for changes in the normal lymph node architecture and cell characteristics. Other procedures used in evaluating lymphomas include blood tests, X-ray, computerized tomography (CT) scan, magnetic resonance imaging (MRI) and bone marrow biopsy.

Many cancers including lymphomas are also being characterized by unique molecular features or inappropriate expression of certain molecules in various malignant cells (e.g., the bcl-2 gene rearrangement found in follicular lymphoma). These molecules thus serve as markers for a particular cancer or lymphoma. Regardless of the procedures used, the ability to accurately determine the presence of a specific lymphoma is quite useful for accurate diagnosis and safe and effective treatment of the lymphoma. Identification of molecules expressed at particular stages of lymphoid cell differentiation or activation can serve as markers useful in diagnosis and treatment of various lymphomas.

### SUMMARY OF THE INVENTION

The present invention discloses that both CDK9 and CYCLIN T1 are expressed in various malignant lymphocytes and lymphomas. In one aspect, the present invention discloses that both CDK9 and CYCLIN T1 proteins are expressed in precursor B and T cells. In peripheral lymphoid tissues, germinal center cells and scattered B and T cell blasts in interfollicular areas express CDK9/CYCLIN T1, while mantle cells, plasma cells and small resting T lymphocytes display no expression of either molecule. The present invention is thus discloses that CDK9/CYCLIN T1 expression is thus related to particular stages of lymphoid differentiation/activation.

In another aspect, the present invention discloses that CDK9 and cyclin T1 complex in malignant lymphomas is highly expressed in lymphomas derived from precursor B and T cells, from germinal center cells, such as follicular lymphomas and from activated T cells, (i.e. anaplastic large cell lymphomas), and Hodgkin and Reed-Sternberg cells of classical Hodgkin lymphoma. Diffuse large B-cell, Burkitt lymphomas and peripheral T cell lymphomas (T-cell lymphoproliferative disorders), showed a wide range of values. No expression of CDK9/CYCLIN T1 is seen in mantle cell and marginal zone lymphomas.

In still another aspect, the present invention discloses that an imbalance in CDK9/CYCLIN T1 mRNA ratio can be used to diagnose certain lymphomas. The imbalance is due to over expression of CDK9 mRNA as compared to the CYCLIN T1 mRNA. Specifically, at RNA level, an imbalance in CDK9/CYCLIN T1 ratio is found in follicular lymphoma, diffuse large B cell lymphomas with germinal center phenotype, and in the cell lines of classical Hodgkin's lymphomas, Burkitt's lymphomas and anaplastic large cell lymphoma in comparison with reactive lymph nodes.

Accordingly, in an embodiment of the invention, a method for determining presence of lymphoma, which is neither mantle cell lymphoma nor marginal zone lymphoma, in a human patient is provided. It requires assaying a sample such as bone marrow, thymus, spleen, lymph nodes, lymph or lymphocytes taken from the lymphatic system of the patient to determine expression of CDK9 and/or CYCLIN T1 protein. The presence of CDK9 and/or CYCLIN T1 proteins in the sample is indicative of a lymphoma in the patient. Lymphoma is one resulting from the expression of CDK9 and CYCLIN T1 in precursor T cells, precursor B cells, germinal center cells, activated T cells or Reed-Sternberg cells (which are very large, abnormal B-cells.

In another embodiment, a method of evaluating a clinical outcome (after chemo and/or radiation treatment) for a patient suffering from lymphoma is provided. It invloves measuring the levels of CDK9 and/or CYCLIN T1 expression in cells in a clinical specimen obtained from the patient and comparing the levels of expression against a set of reference expression levels (e.g., expression levels in normal, non-malignant lymphocytes or expression levels in tonsil cells of a healthy individual) wherein an increase or decrease in the level of expression of CDK9 and/or CYCLIN T1 is indicative of clinical outcome for the patient.

### BRIEF DESCRIPTION OF THE FIGURES

FIG. 1 shows immunohistochemical analysis of CDK9 expression in reactive lymph node (a) and (b); in follicular lymphoma (FL) (c); in DLBCL (d); in cHL (e); ALCL (f). (Original magnification (a) 100×; (b and c) 200×, (d, e and f) 400×).

FIG. 2 shows percentages of CDK9 positive cells in different lymphoma types.

FIG. 3 shows CDK9 and CYCLIN T1 mRNA expression in reactive lymph nodes, malignant lymphomas and in cell lines. (a) CDK9 and CYCLIN T1 mRNA levels in reactive germinal center and mantle cells compared to that observed in MCL, FL and DLBCL; (b) CDK9 and CYCLIN T1 mRNA levels in two different groups of DLBCL. In group 1, DLBCL with germinal center-like (GC-like) phenotype; in group 2 DLBCL with non GC-like phenotype (defined by expression of CD10⁻, Bcl-6⁻); (c) CDK9 and CYCLIN T1 mRNA levels in cHL, BL and ALCL cell lines.

FIG. 4 shows Western blot analysis of two MCL samples (lanes 1 and 2) (a) for CDK9 and (b) for CYCLIN T. Jurkatt cells: positive control.

### DETAILED DESCRIPTION OF THE INVENTION

The present invention relates to methods for determining presence of cancer by analyzing the expression of CDK9 and CYCLIN T1 in lymphoid tissue.

CDK9 is a member of the CDC2-like family of kinases. This kinase, also referred to as PITALRE, was cloned by PCR using degenerate oligonucleotide primers derived from sequences that are conserved in other CDC2-related kinases (Grana et al., 1994, Proc Natl Acad Sci USA, 91:3834-3838). All the members of this family of kinases are characterized by a PSTAIRE or PSTAIRE-like amino acid sequence near the amino terminus of the protein. CDK9 may complex with various members of the T family of cyclins (T1, T2a and T2b) as well as CYCLIN K (Fu et al., 1999, J Biol Chem, 274:34527-34530; Peng et al., 1998, Genes Dev, 12:755-762), while CYCLIN T1 plays the most important role in regulating CDK9 activity. The induction of CYCLIN T1 expression appears to occur through a post-transcriptional mechanism (Herrmann et al., 1998, J Virol, 72:9881-9888) suggesting that CYCLIN T1 is the limiting element of the complex.

The CDK9/CYCLIN T1 complex seems to be required for the differentiation process of several cell types. Overproduction of CDK9/CYCLIN T2 complex enhances MyoD function and promotes myogenic differentiation, while inhibition of CDK9 kinase activity by a dominant negative form prevents the activation of the myogenic program (Simone et al., 2002, Oncogene, 21:4137-4148). The CDK9 and CYCLIN T1 expression also increases in neurons during differentiation, while no variation of their expression level is observed during astrocyte maturation, suggesting that CDK9 involvement in differentiation may vary according to cell types and may depend on different stimuli (De Falco et al., 2002, Cancer Biol Ther, 1:342-347).

In the following description of specific working examples, evaluation of CDK9 and CYCLIN T1 expression in lymphoid tissues is provided to determine that these molecules are involved in the activation and differentiation of lymphoid cells. It also provides an analysis of the expression of CDK9 and CYCLIN T1 in B and T cell lymphomas to show that their expression level is correlated with neoplastic transformation. The abbreviation used for specific lymphomas are as follows: B-LBL: precursor B-cell lymphomas; T-LBL: precursor T-cell lymphoma; MCL: mantle cell lymphoma; MZL: marginal zone lymphoma; FL: follicular lymphoma; DLBCL: diffuse large B cell lymphoma; BL: Burkitt lymphoma; cHL: classical Hodgkin lymphoma; ALCL: anaplastic large cell lymphoma; PTCL: peripheral T-cell lymphoma.

The terms and expressions which have been employed in the description herein are used as terms of description and not of limitation, and there is no intention in the use of such terms and expressions of excluding any equivalents of the features shown and described or portions thereof.

Selection of cases and conventional histology: Twenty reactive lymph nodes, 3 normal thymus, 4 normal bone marrow and 163 lymphoma cases (Table 1) were retrieved from the Department of Human Pathology and Oncology, University of Siena (Italy), the Department of Pathology, “G. Cotugno” Hospital, Naples (Italy) and the Department of Haematology “L.A. Seragnoli”, Bologna (Italy).

Stainings employed for qualitative histological evaluation included haematoxylin and eosin, Giemsa, PAS and Gomori's silver impregnation. Using the immunohistochemical results, two pathologists independently evaluated the cases and established a consensus on diagnosis, based on the WHO Classification. Information on age and sex of patients as well as the site of the biopsies was available. Frozen tissue for molecular analysis was available for five cases of reactive lymph nodes, five cases of MCL, six cases of FL and eight cases of DLBCL.

Immunohistochemistry: Immunophenotyping on paraffin sections was performed using a large panel of antibodies (Table 2) and the ULTRAVISION/AP method (Bioptica, Milan, Italy). Antigen retrieval was performed in 1 mM EDTA (pH 8.0) by heating sections either in a pressure-cooker or a Microwave oven, according to previous experience and depending on the antibody used.

Antibodies for lymphoma immunophenotyping are reported in table 2 and were used at the dilution recommended by manufacturers. Monoclonal anti-CDK9 (sc-13130) and polyclonal anti-CYCLIN T1 (sc-8127) were obtained from SantaCruz, Calif., and used at the dilution of 1:50. In all sections, cells exhibiting positive immunostaining to a given antibody were counted in a randomly chosen high power field (HPF) of lymphoma tissues, and the results were expressed as percentages of all neoplastic cells in those areas. Intra- and inter-observer reproducibility of counts was 95%.

Negative controls were obtained by replacing the primary antibodies with normal mouse/goat serum depending on the antibody used. Normal human tonsils served as positive controls.

Double staining: Double staining for CD3, CD20, CD79a, CD34 and CD68 in combination with CDK9 and CYCLIN T1 was performed on selected specimens of reactive lymph nodes and bone marrow. Paraffin sections of reactive lymph node were dewaxed and rehydrated in the usual way. All sections were incubated in a microwave oven (750W) in Tris EDTA buffer pH 9 for 2 minutes and placed in TBS for 5 minutes. Endogenous peroxidase was blocked using Peroxidase Blocking Reagent (DAKO, UK) for 20 minutes. The sections were then incubated with anti-CDK9 antibody and CYCLIN T1 antibody, both at a dilution of 1:50. After washing in TBS, the slides were incubated with anti-mouse EnVision™ HRP reagent (DAKO, UK). The slides were developed using the DAB substrate provided with the EnVision™ System kit.

Anti-CD3, CD20, CD79a, CD34 and CD68 antibodies (see Table 2) were then applied to the sections at appropriate dilutions. After washing in TBS, the antibodies were detected by anti-mouse EnVision™ AP reagent (DAKO, UK). The slides were developed using the Vector Blue Substrate Kit (Vector Labs, UK).¹¹ The sections were washed in tap water and mounted in Aquamount (Merck, Germany). All primary and secondary antibody incubations lasted 30 minutes at room temperature.

RNA isolation from whole tissues: Sections from 5 MCL cases and 8 DLBCL cases were produced using sterile blades, then homogenized in Tri reagent. Total RNA was extracted according to the manufacturer's instructions (Invitrogen, CA).

Laser Capture Microdissection and RNA extraction: Germinal center and mantle zone areas from five reactive lymph nodes and tumor cell areas from six cases of FL were identified based on H&E stained sections and isolated by Laser Capture Microdissection (LCM) (Arcturus PixCell II™, MWG-BIOTECH, Florence, Italy).

Before sectioning, the cryostat was wiped down with 100% ethanol to avoid cross-contamination, and a fresh disposable blade was used for each case. 5-6 μm thick sections were placed at room-temperature onto Silane Prep Slides (Sigma, Saint Louis, Mo., USA); the slides were stored in a slide box on dry ice until cutting of the remaining sections was completed. A Histogene™ staining kit (Arcturus PixCell II™ MWG-BIOTECH, Florence, Italy) was used to prepare the tissue for LCM, following the manufacturer's recommendations.

Microdissected cells were immediately processed using the PicoPure™ RNA isolation kit (Arcturus, MWG Biotech, Florence, Italy). Briefly, the Capsure™ transfer film carrier was placed directly onto a standard microcentrifuge tube containing 10 μl extraction buffer. The tube was then placed upside-down at 42° C. for 30 minutes so that the extraction buffer was in contact with the tissue on the cap; the remaining extraction procedure was performed according the manufacturer's instructions.

Cell lines: As fresh lymphoma tissue was not available for BL, cHL and ALCL, we decided to use cell lines for RNA extraction. Three ALCL cell lines (Fe—PD, OHNE OMNE, 299), one BL cell line (Daudi) and two cHL cell lines (L1236, L428) were obtained from Institut für Pathologie Universitätsklinikum, Benjamin Franklin Freie Universitat, Berlin, Germany. The RNA was subjected to DNase treatment and then used for RT-PCR.

RT-PCR: For RT-PCR analysis, 10 μl isolated RNA was mixed with 15 μl reverse transcriptase master mixture for the synthesis of cDNA. Reverse transcription was carried out for 1 hour at 42° C., using AMV (Promega) in the presence of RNAsin (Promega). Real-time PCR was performed using the apparatus (LightCycler) supplied by Roche. The DNA master SYBR green 1 kit (Roche Diagnostics, Germany) was used following the manufacturer's instructions. CDK9 and CYCLIN T1 were normalized to G3PDH (primers for CDK9: forward, 5′-ACGGCCTCTACTACATCCACA-3′ (SEQ ID NO: 1) and reverse, 5′-GCTGCGGGTCCACATCTCTGC-3′ (SEQ ID NO: 2); CYCLIN T1 oligonucleotide sequences were: forward, 5′-AAACCAGAGGAGATAAAAATG-3′ (SEQ ID NO: 3) and reverse, 5′-GAATGAGAGTGCTTGTGTGAG-3′ (SEQ ID NO: 4). Primer sequences for G3PDH have been previously described.¹³ To amplify the housekeeping gene G3PDH, the same RNA of each sample was used. All experiments were performed in triplicate.

Western blotting: Five fresh samples of MCL were homogenized in EBC buffer (50 mM Tris-HCl pH. 8.0, NaCl 120 mM, 0.5% NP40 and fresh protease inhibitors). Protein concentration was estimated using the Bradford assay (Biorad, Calif.). 50 μg of protein extract was loaded on a 10% SDS-PAGE and separated. Western blotting (WB) was performed using monoclonal anti-CDK9 (sc-13330, Santa Cruz, Calif.) at a dilution of 1:500 and a polyclonal anti-CYCLIN T1 (sc-8127, Santa Cruz, Calif.), at a dilution of 1:500. A Jurkatt cell line was used as a positive control. All experiments were performed in triplicate.

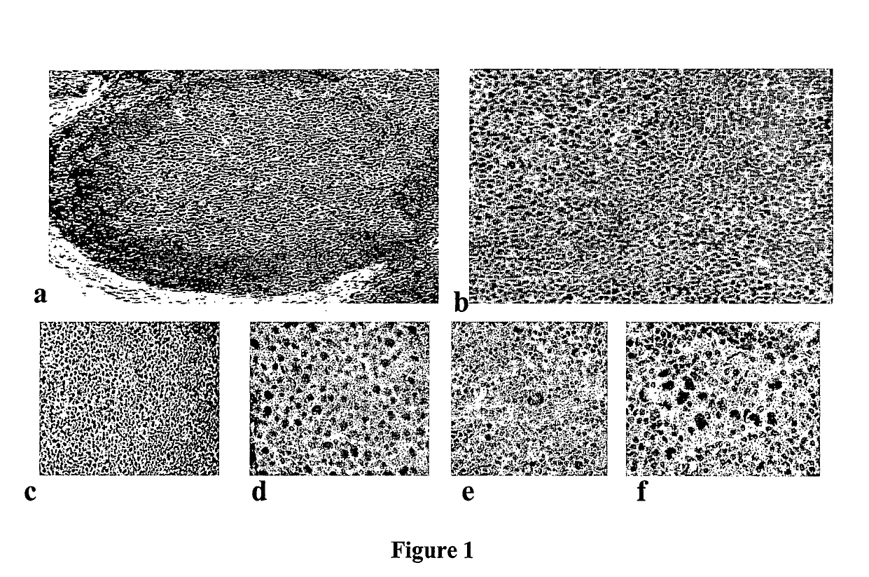

The expression of CDK9 and CYCLIN T1 was determined by immunohistochemistry and both showed expression in lymphoid cells as shown by the nuclear staining pattern. The CDK9 and CYCLIN T1 expression was found mainly in the thymic lymphoid population of the outer cortex beneath the capsule, as well as in neoplastic T cell precursors. In the bone marrow, CDK9 and CYCLIN T1 expression was present in more immature cells of lymphoid and myeloid derivation. CDK9 and CYCLIN T1 nuclear staining was found in most of the cells positive for CD34 (stem cell, pro-B cells), in a small proportion of CD20 positive cells, probably representing pre-B cells, in CD68 positive myeloblasts and in megacarioblasts. Tumors derived from precursor B cells also had nuclear staining for both CDK9 and CYCLIN T1. In peripheral lymphoid tissues, CDK9 and CYCLIN T1 expression was found in germinal center cells (GC), particularly in centroblasts (FIG. 1a and 1b). The mantle cells were consistently negative in all the cases examined. Scattered B and T cell blasts in the interfollicular areas also expressed CDK9 and CYCLIN T1 as demonstrated by double staining with CD20 and CD3 antibodies respectively. Resting small B and T lymphocytes were negative. Macrophages and mature plasmacells in the medullary sinuses did not display any reactivity with CDK9 and CYCLIN T1 antibodies. Among lymphomas derived from peripheral B and T lymphoid cells, FL (FIG. 1 c) and ALCL (FIG. 1 f) showed above 40% of neoplastic cells positive for both proteins. Hodgkin and Reed-Sternberg cells of classical Hodgkin lymphoma also showed a strong nuclear staining for both proteins (FIG. 1e). DLBCL (FIG. 1 d), BL, and PTCL (not shown) demonstrated great variability, with a range of values from 0 to 100%. DLBCLs were further classified into GC-like and non-GC like according to immunohistochemical expression of CD10, BCL6 and MUM-1 (table 3). Interestingly, a correlation between the percentage of CDK9 and CYCLIN T1 positive cells and the expression of germinal center markers, such as BCL6 (r=0.81; p<0.001) and CD10 (r=0.83; p<0.001), was found in DLBCL (data not shown), while there was no correlation with MUM1. No expression of CDK9 and CYCLIN T1 was detected in MZL or MCL.

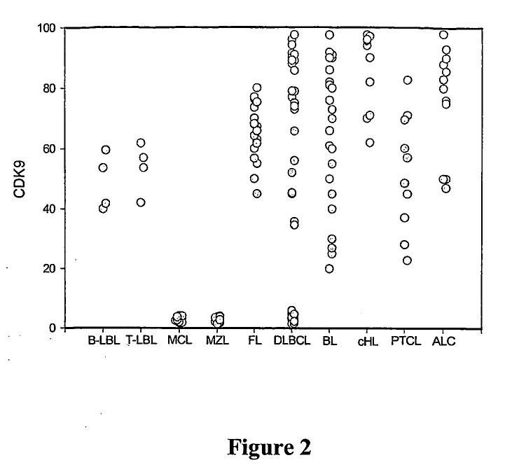

The results of CDK9 expression in malignant lymphomas are summarized in FIG. 2. The results obtained for CYCLIN T1 were closely correlated with those of CDK9 (data not shown).

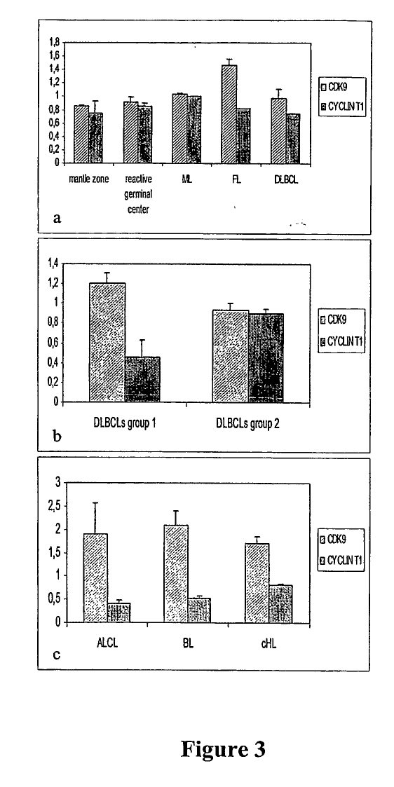

The mRNA expression of CDK9 and CYCLIN T1 in reactive lymph nodes, in some samples of malignant lymphomas and in cell lines was analyzed by RT-PCR. The results are summarized in FIG. 3 (a, b and c).

In microdissected reactive germinal center and mantle cells, comparable levels of CDK9 and CYCLIN T1 mRNA were observed, with a ratio 1:1 although no expression of either molecule was detectable at protein level by immunohistochemistry in normal mantle cells.

CDK9 and CYCLIN T1 mRNA expression levels varied in the tumor samples analyzed, depending on the lymphoma type. In MCL, CDK9 and CYCLIN T1 mRNA were expressed at the same levels as in their normal counterparts (FIG. 3a). In contrast, in FL CDK9 mRNA was over-expressed when compared to reactive germinal centers, while no difference in terms of CYCLIN T1 expression was observed between reactive and neoplastic germinal centers.

In DLBCL a heterogeneous situation was found: average values of CDK9 and CYCLIN T1 expression in all cases indicated a slight increase in the CDK9 mRNA level. However, the DLBCLs with GC-like phenotype showed a dramatic imbalance in the CDK9/CYCLIN T1 ratio, which resembled the situation observed in FL (FIG. 3b).

In the cHL, BL, and ALCL cell lines analyzed, an over-expression of CDK9 was also observed while CYCLIN T1 was poorly expressed, leading again to an imbalance of the CDK9/CYCLIN T1 ratio (FIG. 3c).

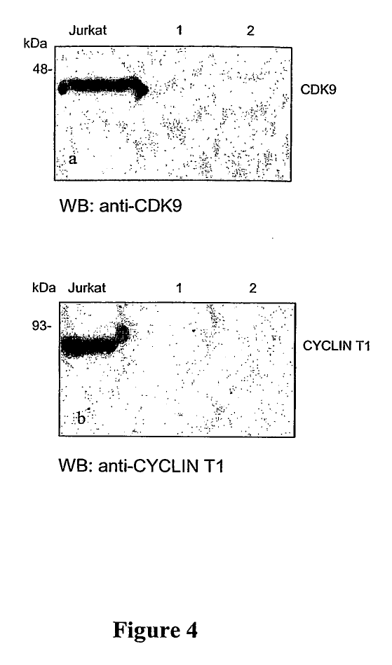

Since normal and neoplastic mantle cells showed CDK9 and CYCLIN T1 mRNA expression similar to germinal center cells, without immunohistochemical protein expression, Western Blot anlysis was carried out in five cases of MCL where frozen tissue was available. In all MCL cases, CDK9 and CYCLIN T1 expression was almost undetectable by Western blotting, as compared to protein expression in a Jurkatt cell line (FIGS. 4a and b).

In the present invention, it has been shown that CDK9/CYCLIN T1 is involved in the differentiation/activation program of B and T lymphocytes. The CDK9 and CYCLIN T1 protein expression varies considerably according to lymphoid cell types. It was present in precursor B and T cells, while in peripheral lymphoid tissues it was consistently detectable at the highest level in antigen-challenged germinal center B cells (centroblasts) before differentiation into plasma or memory B cells. In addition, scattered B and T cell blasts in the interfollicular areas also expressed CDK9 and CYCLIN T1 whereas mantle cells and small resting T lymphocytes displayed no expression of either molecule. These results show that CDK9/CYCLIN T1 is expressed in lymphoid cells at particular stages of their differentiation/activation program. In addition, the expression of these proteins is shown herein to be cell cycle related, since they are mainly found in proliferating cells, such as precursor B and T cells, germinal center cells and immunoblasts. This finding is in line with the experimental evidence that peripheral blood lymphocytes enter and progress through the cell cycle following activation by PMA and PHA and the expression of CDK9/CYCLIN T1 is simultaneously dramatically upregulated (Herrmann et al., 1998, J Virol, 72:9881-9888). However, the expression of CDK9/CYCLIN T1 is not growth and/or cell cycle related in other cell types. In both skeletal muscle and neural cell lines CDK9 kinase activity is higher at the end of differentiation than during asynchronous growth, although at least in C2C12 cells the level of activity appears to be highest before terminal differentiation is reached (MacLachlan et al., 1998, J Cell Biochem, 71:467-478; Sano et al., 2002, Nat Med, 8:1310-1317; Napolitano et al., 2002, J Cell Physiol, 192:209-215).

The immunohistochemical expression of CDK9 and CYCLIN T1 complex in malignant lymphomas seems to reflect their cellular origin, as it is highly expressed in lymphomas derived from precursor T and B cells, germinal center cells (FL) and from activated T cells (i.e. ALCL). The finding of CDK9 and CYCLIN T1 expression in Hodgkin and Reed-Stemberg cells of classical HD is also in line with their derivation from GC or post-GC cells. DLBCL, BL and PTCL, among T-cell lymphoproliferative disorders, showed a wide range of values, probably reflecting their heterogeneity in the cell of origin. In contrast, no expression of CDK9 and CYCLIN T1 was detected in MZL or MCL by immunohistochemistry. As a result, immunostaining is suitable to identify lymphoid neoplasias derived from stages where CDK9 and CYCLIN T1 are constitutively highly expressed.

Unexpectedly, the CDK9 and CYCLIN T1 mRNA expression pattern is not completely in harmony with the protein expression profile as detected by immunohistochemistry. The levels of mRNA of CDK9 and CYCLIN T1 in normal and neoplastic mantle cells are similar when compared to reactive GC cells, although in mantle cells no protein expression was detectable for either molecule. Undetectable protein expression in cells where CDK9 and CYCLIN T1 are transcribed suggests a blockage at post-transcriptional level or a rapid turnover of the proteins in cells at particular stages of differentiation in which their function is not required. In addition, CDK9 was strongly over-expressed in neoplastic GC cells of FL, whereas CYCLIN T1 was not affected. The ratio of CDK9 and CYCLIN T1 in DLBCL with GC-like phenotype was similar to that in FL. Similarly cHL, BL, and ALCL cell lines showed low CYCLIN T1 mRNA in the presence of enhanced levels of CDK9 mRNA. These findings show that neoplastic transformation in lymphoid tissues may be associated with an imbalance in the CDK9/CYCLIN T1 ratio. Due to the importance of CDK9/CYCLIN T1 complex in transcription and differentiation, its imbalance may be involved in the deregulation of activated transcription mediated by not yet identified transcription factors. The pattern of CDK9 and CYCLIN T1 distribution has been found to be altered in cells treated with transcription inhibitors. Transient expression of CYCLIN T1 deletion mutants indicated its crucial role in transcription (Herrmann et al., 2001, J Cell Sci, 114(Pt8): 1491-1503).

The foregoing examples describe methods for determining expression of CDK9 or CYCLIN T1 protein or RNA as a possible indication of cancer. As was indicated, supra, these genes are expressed in malignant lymphocytes, thereby enabling the skilled artisan to utilize these for, e.g., assaying for lymphomas.

Any conveniently available tissue or liquid sample from a patient (human patient) can be used for measurement of gene expression levels. In particular embodiments, the samples being analyzed are bone marrow, thymus tissue sample, spleen tissue sample, lymph nodes, lymph and/or lymphocytes. The sample is derived from biopsy. In one embodiment, the sample is one, which is readily and easily available via minimally invasive methods. Methods for preparing the sample for gene expression analysis are well known in the art, and can be carried out using commercially available kits.

The gene expression levels used in the methods of the invention can be measured by any method now known or that is devised in the future that can provide quantitative information regarding the levels to be measured. The methods preferably are highly sensitive and provide reproducible results. In one embodiment, methods based upon nucleic acid amplification technologies are used. In particular, methods based upon the polymerase chain reaction (PCR) and related amplification technologies, such as NASBA and other isothermal amplification technologies, may be used. More particularly, so called RT-PCR methods using reverse transcription of mRNA of CDK9 or CYCLIN T1 genes followed by amplification of the resulting cDNA are contemplated.

The determination of expression can also be carried out via, e.g., determination of transcripts of CDK9 and/or CYCLIN T1 gene or genes, via nucleic acid hybridization. In a preferred embodiment, one determines presence of a transcript of CDK9 or CYCLIN T1 gene by contacting a sample with a nucleic acid molecule which specifically hybridizes to the transcript. The hybridization of the nucleic acid molecule to a target is indicative of expression of a CDK9 or CYCLIN T1 gene, and of the possibility of cancer. Preferably, this is done with two primer molecules, as in a polymerase chain reaction. Determination of expression of CDK9 or CYCLIN T1 genes in the context of these assays also is a part of the invention.

Alternate assays are also part of the invention. These include electrophoresis or immunophenotyping, immunoblotting, immunohistochemistry or immunofluorescence microscopy of the sample with one or more selected antibodies.

Such assays can be carried out in any of the standard ways one determines antibodies, such as by contacting the sample with an amount of protein or proteins, and any additional reagents necessary to determine whether or not the antibody binds. One approach involves the use of immobilized protein, where the protein is immobilized in any of the standard ways known to the art, followed by contact with the sample and then, e.g., anti-IgG, anti-Fc antibodies, and so forth. Conversely, presence of CDK9 and/or CYCLIN T1 protein can also be determined, using antibodies in the place of the proteins of the above described assays.

In addition to the correlation of CDK9 or CYCLIN T1 expression with specific lymphomas, various therapeutic methods and compositions useful in treating conditions associated with abnormal CDK9 or CYCLIN T1 expression are also part of the present invention. Abnormal CDK9 or CYCLIN T1 expression” in this context may mean expression per se, or levels which differ from those in a normal individual, i.e., they may be lower or higher.

The invention envisions therapeutic approaches such as the use of antisense molecules to inhibit or block CDK9 or CYCLIN T1 expression in malignant lymphocytes. These antisense molecules are oligonucleotides which hybridize to the nucleic acid molecules and inhibit their expression. Preferably these are 17-50 nucleotides in length. These antisense oligonucleotides are preferably administered in combination with a suitable carrier, such as a cationic liposome.

Other therapeutic approaches include the administration of CDK9 or CYCLIN T1 proteins per se, one or more antigenic peptides derived therefrom, as well as so-called polytopic vaccines or inhibitors of CDK9 or CYCLIN T1 proteins. The polytopic vaccines include a plurality of antigenic peptides, untied together, preferably by linker sequences. The resulting peptides may bind to either MHC-Class I or Class II molecules. These proteins, peptides, or polytopic vaccines may be administered in combination with an appropriate adjuvant. They may also be administered in the form of genetic constructs which are designed to permit expression of the protein, the peptide, the polytopic structures, etc.

One can formulate the therapeutic compositions and approaches described herein The amount of agent administered and the manner in which it is administered will vary, based on the condition being treated and the individual. Standard forms of administration, such as intravenous, intradermal, subcutaneous, oral, rectal and transdermal administration can be used. With respect to formulations, the proteins and or peptides may be combined with adjuvant and/or carriers. Other aspects of the invention will be clear to the skilled artisan and need not be reiterated herein.

When the nucleic acid approach is utilized, various vectors, such as Vaccinia, retrovirus or adenovirus based vectors can be used. Any vector useful in eukaryotic transfection, such as in transfection of human cells, can be used. These vectors can be used to produce, e.g., cells such as dendritic cells which present relevant peptide/MHC complexes on their surface. The cells can then be rendered non-proliferative prior to their administration, using standard methodologies.

The present invention also contemplates molecular detection of residual tumor cells following lymphoma therapy. Although a complete clinical remission can often be achieved with chemotherapy for patients with lymphoma, relapses still occur. Residual tumour cells probably have survived therapy and account for subsequent disease relapse. These may then account for subsequent disease relapse. Patients receiving high dose chemotherapy with autologous stem cell rescue may also relapse as a result of occult tumour contamination in the autologous stem cell or bone marrow given. The methods of the present invention may also be used to assess the effectiveness of bone marrow purging if it is performed before a transplant.

All publications and references, including but not limited to patent applications, cited in this specification, are herein incorporated by reference in their entirety as if each individual publication or reference were specifically and individually indicated to be incorporated by reference herein as being fully set forth. While this invention has been described with a reference to specific embodiments, it will be obvious to those of ordinary skill in the art that variations in these methods and compositions may be used and that it is intended that the invention may be practiced otherwise than as specifically described herein. Accordingly, this invention includes all modifications encompassed within the spirit and scope of the invention as defined by the claims.

TABLE 1
Histological diagnosis of Lymphoma cases

| Diagnosis | Number of cases |
|---|---|
| B-LBL | 4 |
| T-LBL | 4 |
| MCL | 12 |
| MZL | 12 |
| FL | 20 |
| DLBCL | 35 |
| BL | 46 |
| CHL | 10 |
| ALCL | 12 |
| PTCL | 10 |

B-LBL: precursor B-cell lymphomas;
T-LBL: precursor T-cell lymphoma;
MCL: mantle cell lymphoma;
MZL: marginal zone lymphoma;
FL: follicular lymphoma;
DLBCL: diffuse large B cell lymphoma;
BL: Burkitt lymphoma;
cHL: classical Hodgkin lymphoma;
ALCL: anaplastic large cell lymphoma;
PTCL: peripheral T-cell lymphoma.

TABLE 2
Monoclonal antibodies used for diagnosis of Lymphoma cases

| Antibody | Source | Molecule Identified |
|---|---|---|
| L26 | Neomarkers | CD20 |
| Anti-CD79a | Dako | CD79a |
| Anti-CD3 | Neomarkers | CD3 |
| Anti-CD10 | Neomarkers | CD10 |
| Anti-Bc12 | Dako | Bc12 |
| Anti-CD34 | Dako | CD34 |
| Anti-CD68 | Dako | CD68 |
| Anti-TdT | Neomarkers | TdT |
| Anti-Bc16 | Dako | Bc16 |
| MUM1 | Dako | MUM1-IRF4 |
| Anti-CD5 | Neomarkers | CD5 |
| ALKc | Novocastra | NPM-ALK |
| Cyclin D | Neomarkers | Cyclin D |
| Anti-CD8 | Dako | CD8 |
| Anti-CD56 | Neomarkers | CD56 |
| Anti-CD4 | Neomarkers | CD4 |
| Anti-CD23 | Immunotech | CD23 |

TABLE 3
Sub-classification of DLBCL into GC-like and non GC-like.

| | CD10 | Bc16 | MUM1 | % |
|---|---|---|---|---|
| GC like | + | +/− | −/+ | 42.6 |
| GC like | − | + | − | 3.2 |
| non-GC like | − | +/− | + | 49.1 |

MUM1 expression was seen in 35.4% of GC cases (1 case with CD10 alone and 10 cases with both CD10 and BCL6).

What is claimed is:

1. A method for determining presence of lymphoma, which is neither mantle cell lymphoma nor marginal zone lymphoma, in a subject comprising assaying a sample taken from the lymphatic system of the subject to determine expression of CDK9 or CYCLIN T1 protein, wherein presence of CDK9 or CYCLIN T1 protein in the sample is indicative of said lymphoma in the subject.

2. The method of claim 1, wherein the sample is bone marrow, thymus, spleen, lymph nodes, lymph or lymphocytes.

3. The method of claim 1, wherein said lymphoma is one resulting from the expression of CDK9 and CYCLIN T1 in precursor T cells, precursor B cells, germinal center cells, activated T cells or Reed-Sternberg cells.

4. The method of claim 1, wherein said lymphoma is neither mantle cell lymphoma nor marginal zone lymphoma.

5. The method of claim 1, wherein said lymphoma is precursor B-cell lymphoma.

6. The method of claim 1, wherein said lymphoma is precursor T-cell lymphoma.

7. The method of claim 1, wherein said lymphoma is follicular lymphoma.

8. The method of claim 1, wherein said lymphoma is diffuse large B cell lymphoma.

9. The method of claim 1, wherein said lymphoma is Burkitt lymphoma.

10. The method of claim 1, wherein said lymphoma is classical Hodgkin lymphoma.

11. The method of claim 1, wherein said lymphoma is anaplastic large cellymphoma.

12. The method of claim 1, wherein said lymphoma is peripheral T-cell lymphoma.

13. The method of claim 1, wherein the assaying comprises, immunophenotyping, immunoblotting, immunohistochemistry or immunofluorescence microscopy of the sample with one or more selected antibodies.

14. A method for determining presence of lymphoma in a human patient, comprising assaying a sample taken from the lymphatic system of the human patient to determine mRNA levels of CDK9 and CYCLIN T1, wherein an imbalance in CDK9/CYCLIN T1 mRNA ratio with increased levels of CDK9 as compared to CYCLIN T1 is indicative of a lymphoma.

15. The method of claim 14, wherein said lymphoma is selected from the group consisting of: follicular lymphoma, diffuse large B cell lymphomas, classical Hodgkin's lymphoma, Burkitt's lymphoma and anaplastic large cell lymphoma.

16. The method of claim 14, wherein the sample is bone marrow, thymus, spleen, lymph nodes, lymph or lymphocytes.

17. The method of claim 14, wherein said lymphoma is one resulting from the expression of CDK9 and CYCLIN T1 in precursor T cells, precursor B cells, germinal center cells, activated T cells or Reed-Sternberg cells
